(19) United States
(12) Patent Application Publication
(10) Pub. No.: US 2010/0286533 A1
(43) Pub. Date: Nov. 11, 2010

(54) Biosignal-detecting apparatus and method thereof

(75) Inventors: Mi Hee LEE, Yongin-si (KR); Kun Soo SHIN, Seongnam-si (KR); Soo Hyun BAE, Seoul (KR); Jong Pal KIM, Seoul (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/976,075

(22) Filed: Oct. 19, 2007

(30) Foreign Application Priority Data
Nov. 27, 2006 (KR) 10-2006-0117509

(51) Int. Cl. A61B 5/0205

(52) U.S. Cl. 600/484

(57) ABSTRACT

A method of detecting a biosignal includes; measuring a biosignal from a user; band pass filtering the biosignal to each of a first frequency band and/or a second frequency band, and detecting a first frequency band signal and/or a second frequency band signal; calculating a PSD of the first frequency band signal and detecting a frequency band signal with a maximum PSD in the first frequency band as a heartbeat signal of the user; and calculating a PSD of the second frequency band signal and/or detecting a frequency band signal with a maximum PSD in the second frequency band as a respiration signal of the user.

### CROSS-REFERENCE TO RELATED APPLICATION

This application claims the benefit of Korean Patent Application No. 10-2006-0117509, filed on Nov. 27, 2006, in the Korean Intellectual Property Office, the disclosure of which is incorporated herein by reference.

### BACKGROUND

1. Field

The embodiments relate to an apparatus and method of detecting a biosignal, and more particularly, to an apparatus and method of detecting a biosignal, which can measure a biosignal from a user, band pass filter the measured biosignal to each of a heartbeat frequency band and a respiration frequency band, and calculate a power spectrum density (PSD) of the band pass filtered signal and thereby, can detect a heartbeat signal and a respiration signal of the user from the biosignal.

2. Description of the Related Art

Currently, many people are seeking a well-being life, and also trying to be healthy. Also, various types of well-being industries are being developed due to a well-being popularity. A heartbeat/respiration measurement apparatus for measuring a user's heart rate or respiration rate to determine the user's health status may be included in the well-being industries.

A conventional heartbeat/respiration measurement apparatus attaches a sensor onto a user's body and measures the user's heart rate and respiration rate via the sensor. However, when the sensor is attached onto the user's body, the user may not freely move, and noise may frequently occur due to the user's motion.

Accordingly, an apparatus capable of wirelessly measuring a user's heart rate and respiration rate in a close range using a radar without attaching a sensor onto the user's body is being utilized. Specifically, the apparatus may transmit a radar signal to the user's body via a radio frequency (RF) sensor, and the like, and detect the user's heartbeat signal and respiration signal using a Doppler shift of a signal that is reflected from the user. In this case, since the user's heart rate and respiration rate may be wirelessly measured without attaching the sensor onto the user's body, the user may readily perform a measurement.

Also, it is very important how accurately and effectively the apparatus may detect the user's heartbeat signal and respiration signal from the signal that is reflected from the user. Specifically, the reflected biosignal of the user generally includes noise due to the user's motion or various types of noise due to the user's heartbeat signal or respiration signal. Accordingly, it is very important to eliminate the various types of noise and thereby detect an accurate heartbeat signal and respiration signal from the biosignal.

A method of detecting a heartbeat signal and a respiration signal according to a conventional art includes a method of detecting a heartbeat signal and a respiration signal by predetermining a reference signal. Specifically, when a reference signal of each of a heartbeat signal and a respiration signal to be detected is predetermined, and then the reference signal is eliminated from the biosignal, only a noise signal remains. Also, when the noise signal is eliminated from the biosignal, the heartbeat signal and the respiration signal may be detected.

However, in this case, the reference signal should include every possible frequency band signal. Therefore, features about the heartbeat signal and the respiration signal should be similar to each other for each user and thus the reference signal may not be readily predetermined. Also, since a contact-type signal, acquired by attaching a sensor onto a user's body, is not measured, an amplitude and a phase of a signal may frequently change depending on a distance between an RF sensor and the user. Accordingly, a predetermined adaptive filter coefficient should be set to compensate for the change in the amplitude and the phase.

Accordingly, an apparatus capable of more accurately and effectively detecting a user's heartbeat signal and respiration signal using a radar is required.

### SUMMARY

Additional aspects and/or advantages will be set forth in part in the description which follows and, in part, will be apparent from the description, or may be learned by practice of the invention.

An aspect of the embodiment provides an apparatus and method of detecting a biosignal, which can measure a biosignal from a user via a radar, band pass filter the measured biosignal to a predetermined heartbeat frequency band, measure a power spectrum density (PSD) of the band pass filtered signal, and detect a frequency band signal with a maximum PSD as the user's heartbeat signal and thereby can reduce effects of motion artifacts of the user and also can more accurately and readily detect the user's heartbeat signal.

An aspect of the embodiment also provides an apparatus and method of detecting a biosignal, which can measure a biosignal from a user via a radar, band pass filter the measured biosignal to a predetermined respiration frequency band, measure a PSD of the band pass filtered signal, and detect a frequency band signal with a maximum PSD as the user's respiration signal, and thereby can reduce effects of motion artifacts of the user and also can more accurately and readily detect the user's respiration signal.

An aspect of the embodiment also provides an apparatus and method of detecting a biosignal, which can compare a PSD of each of a detected heartbeat signal and respiration signal with a previously measured PSD of each of the heartbeat signal and respiration signal, determine whether the detected heartbeat signal and the respiration signal correspond to accurate signals and thereby can secure a more accurate measurement with respect to the heartbeat signal and the respiration signal.

According to an aspect of the embodiment, there is provided a method of detecting a biosignal, the method including: measuring a biosignal from a user; band pass filtering the biosignal to a first frequency band; and calculating a PSD of the band pass filtered signal and detecting a frequency band signal with a maximum PSD as a heartbeat signal of the user.

According to another aspect of the embodiment, there is provided a method of detecting a biosignal, the method including: measuring a biosignal from a user; band pass filtering the biosignal to a second frequency band; and calculating a PSD of the band pass filtered signal and detecting a frequency band signal with a maximum PSD as a respiration signal of the user.

According to still another aspect of the embodiment, there is provided a method of detecting a biosignal, the method including: measuring a biosignal from a user; band pass filtering the biosignal to each of a first frequency band and a second frequency band, and detecting a first frequency band signal and a second frequency band signal; calculating a PSD of the first frequency band signal and detecting a frequency band signal with a maximum PSD in the first frequency band as a heartbeat signal of the user; and calculating a PSD of the second frequency band signal and detecting a frequency band signal with a maximum PSD in the second frequency band as a respiration signal of the user.

According to yet another aspect of the embodiment, there is provided an apparatus for detecting a biosignal, including: a sensor unit to measure a biosignal from a user; a filter unit to filter band pass the biosignal to each of a first frequency band and a second frequency band, and to detect a first frequency band signal and a second frequency band signal; a PSD calculation unit to calculate PSDs of the first frequency band signal and the second frequency band signal; and a signal detection unit to detect a frequency band signal with a maximum PSD in the first frequency band as a heartbeat signal of the user, and to detect a frequency band signal with a maximum PSD in the second frequency band as a respiration signal of the user.

Additional and/or other aspects and advantages of the embodiment will be set forth in part in the description which follows and, in part, will be obvious from the description, or may be learned by practice of the invention.

### BRIEF DESCRIPTION OF THE DRAWINGS

The above and/or other aspects and advantages of the embodiment will become apparent and more readily appreciated from the following detailed description, taken in conjunction with the accompanying drawings of which:

FIG. 1 is a diagram illustrating a configuration of a biosignal measurement system according to an exemplary embodiment;

FIG. 2 is a block diagram illustrating a configuration of a biosignal detection apparatus according to an exemplary embodiment;

FIG. 3 is a flowchart illustrating a method of detecting a biosignal according to an exemplary embodiment;

FIG. 4 is a flowchart illustrating a method of detecting a biosignal according to another exemplary embodiment; and

FIG. 5 is a flowchart illustrating a method of detecting a biosignal according to still another exemplary embodiment.

### DETAILED DESCRIPTION OF THE EMBODIMENTS

Reference will now be made in detail to exemplary embodiments, examples of which are illustrated in the accompanying drawings, wherein like reference numerals refer to the like elements throughout. The exemplary embodiments are described below in order to explain the present invention by referring to the figures.

A biosignal measurement apparatus according to the embodiment may be included in any one of a mobile communication terminal, a personal digital assistant (PDA), a portable game device, an MPEG audio layer-3 (MP3) player, a personal multimedia player (PMP), a Digital Multimedia Broadcasting (DMB) terminal, and the like. Also, the biosignal measurement apparatus may not be installed in the devices, but may be designed to have a stand-alone configuration.

Hereinafter, exemplary embodiments will be described in detail with reference to the accompanying drawings.

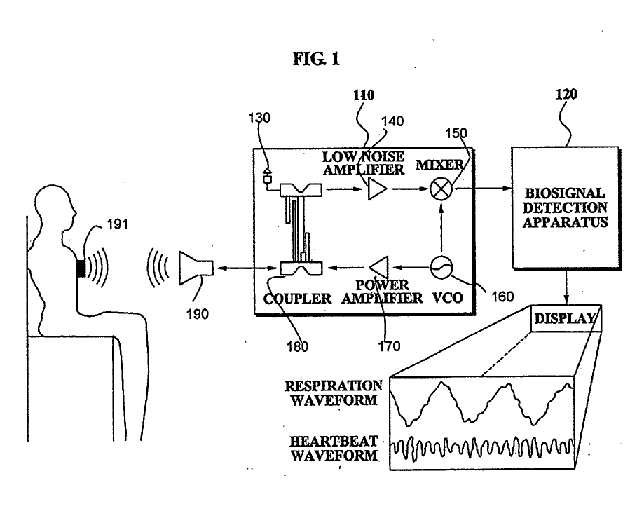

FIG. 1 is a diagram illustrating a configuration of a biosignal measurement system according to an exemplary embodiment.

The biosignal measurement system according to the present exemplary embodiment may include a biosignal detection sensor integrated circuit (IC) 110 and a biosignal detection apparatus 120. The biosignal detection sensor IC 110 may include a coupler, a low noise amplifier 140, a mixer 150, a voltage-controlled oscillator 180(VCO), a power amplifier 170 and an antenna 130.

The biosignal detection sensor IC 110 oscillates a radar signal towards a user's body via the antenna 130. Also, the biosignal detection sensor IC 110 receives the user's biosignal. The biosignal corresponds to a signal which is acquired when the radar signal is reflected from the user's body.

The biosignal received by the biosignal detection sensor IC 110 is transmitted to the biosignal detection apparatus 120. The biosignal detection apparatus 120 may include a filter, an amplifier, and a digital signal processor (DSP) module, which are not shown. The biosignal detection apparatus 120 filters the biosignal to a predetermined band using the filter, and amplifies the filtered signal using the amplifier.

The biosignal detection apparatus 120 performs a predetermined digital signal process with respect to the filtered and amplified signal using the DSP module, and detects the user's heartbeat signal or respiration signal from the biosignal. Also, the biosignal detection apparatus 120 may display the detected heartbeat signal or respiration signal on a predetermined display for the user.

Also, the biosignal detection apparatus 120 may transmit the heartbeat signal or the respiration signal to an external communication terminal or an external server using a predetermined communication module. As shown in FIG. 1, the user may recognize the user's own heart rate, heart rate variability (HRV), and respiration rate using a heartbeat signal waveform or a breathing signal waveform and thereby check the user's health.

The biosignal measurement system shown in FIG. 1 may measure a user's heartbeat signal and/or respiration signal, and also may measure various types of biosignals associated with the user's internal organs. Also, as shown in FIG. 1, the biosignal measurement system may measure the user's biosignal using a Doppler radar sensor that oscillates a radar signal, and also may measure the user's biosignal using a piezoelectric sensor that is attached onto the user's body and a radio frequency (RF) sensor 190 that wirelessly receives a signal from the piezoelectric sensor 191. Specifically, the biosignal measurement system may include any type of sensor, which is widely utilized in the related arts, to measure the user's biosignal.

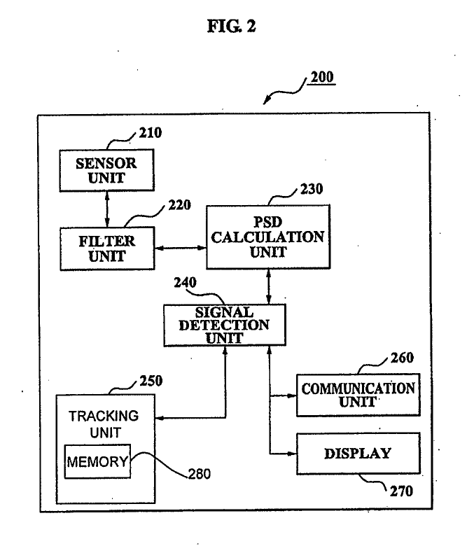

As described above, the biosignal detection apparatus 120 may detect the user's heartbeat signal and/or respiration signal from the biosignal which is measured from the user using the biosignal detection sensor IC 110. Also, the biosignal detection apparatus 120 may detect various types of biosignals associated with the user. However, in the present specification, for convenience of description, a configuration and operation of when a biosignal detection apparatus detects a heartbeat signal or a respiration signal from a user's biosignal will be described. Also, a configuration when a biosignal detection apparatus includes a biosignal detection sensor IC 110 will be described with reference to FIG. 2.

FIG. 2 is a block diagram illustrating a configuration of a biosignal detection apparatus 200 according to an exemplary embodiment.

The biosignal detection apparatus 200 may include a sensor unit 210, a filter unit 220, a power spectrum density (PSD) calculation unit 230, a signal detection unit 240, a tracking unit 250, a communication unit 260, and a display 270.

The sensor unit 210 measures a biosignal from a user. As described with FIG. 1, the sensor unit 210 oscillates a radar signal towards the user's body, and receives the user's biosignal. The biosignal corresponds to a signal which is acquired when the radar signal is reflected from the user's body. To measure the user's biosignal, the sensor unit 210 may include either a Doppler radar sensor, or a piezoelectric sensor and an RF sensor. Also, the sensor unit 210 may include various types of widely-utilized sensors in the related arts in addition to the above-described sensors.

The filter unit 220 band pass filters the biosignal to each of a first frequency band and a second frequency band, and detects a first frequency band signal and a second frequency band signal. Here, the first frequency band corresponds to the user's heartbeat signal frequency band, and the second frequency band corresponds to the user's respiration signal frequency band. Specifically, the filter unit 220 may band pass filter the biosignal to the first frequency band or the second frequency band to detect the user's heartbeat signal or respiration signal.

A human being's normal heart rate may be about from 60 times to 90 times a minute. Also, a human being's normal respiration rate may be about from 12 times to 21 times a minute. Accordingly, a frequency band of a normal heartbeat signal may be estimated to be within the range of about 1.0 Hz to about 1.5 Hz. Also, a frequency band of a normal respiration signal may be estimated to be within the range of about 0.2 Hz to about 0.35 Hz.

Specifically, to detect the user's heartbeat signal from the biosignal, the filter unit 220 may band pass filter the biosignal to the first frequency band of about 1.0 Hz to about 1.5 Hz and thereby detect a first frequency band signal from the biosignal. Also, to detect the user's respiration signal from the biosignal, the filter unit 220 may band pass filter the biosignal to the second frequency band of about 0.2 Hz to about 0.35 Hz, and thereby detect a second frequency band signal from the biosignal.

Here, the frequency band of the heartbeat signal corresponding to the first frequency band and the frequency band of the respiration signal corresponding to the second frequency band may be only an example that is set based on the user's heart rate and respiration rate for convenience of description. Therefore, the first frequency band and the second frequency band may be set to various values depending on a decision of those in the related art.

As described above, to estimate the heartbeat signal or the respiration signal from the biosignal, the first frequency band or the second frequency band may be set by considering the user's heart rate or respiration rate.

Also, the first frequency band and the second frequency band may be set using a predetermined user calibration. Specifically, when the user is not suffering from any illnesses and is at rest, the user's heart rate or respiration rate may be measured, and the first frequency band or the second frequency band may be set by considering the measured heart rate or respiration rate. When there is a plurality of users, the first frequency band or the second frequency band may be set to have a different value for each of the plurality of users.

In this case, the first frequency band or the second frequency band is set by considering each of the plurality of users' unique physical features and thus, a heartbeat signal or a respiration signal according each of the plurality of users' unique physical features may be more accurately detected from the biosignal.

The PSD calculation unit 230 calculates a PSD of the first frequency band. Specifically, the PSD calculation unit 230 calculates the PSD of the first frequency band which is acquired by band pass filtering the biosignal to the first frequency band by the filter unit 220.

Also, the PSD calculation unit 230 calculates a PSD of the second frequency band signal. Specifically, the PSD calculation unit 230 calculates the PSD of the second frequency band signal which is acquired by band pass filtering the biosignal to the second frequency band by the filter unit 220. To calculate the PSD of each of the first frequency band signal and the second frequency band signal, the PSD calculation unit 230 may include a predetermined software module which contains a PSD calculation algorithm.

The signal detection unit 240 detects a frequency band signal with a maximum PSD in the first frequency band as a heartbeat signal of the user, and/or detects a frequency band signal with a maximum PSD in the second frequency band as a respiration signal of the user.

More specifically, the signal detection unit 240 detects the frequency band signal with the maximum PSD in the first frequency band to detect the user's heartbeat signal from the first frequency band signal. As an example, the first frequency band signal may be divided into a 1-1 frequency band signal, a 1-2 frequency band signal, and a 1-3 frequency band signal according to a calculation of the PSD. In this instance, the signal detection unit 240 may compare a PSD of each of the 1-1 frequency band signal, the 1-2 frequency band signal, and the 1-3 frequency band signal, and detect the frequency band signal with the maximum PSD as the user's heartbeat signal.

Also, the signal detection unit 240 detects a frequency band with the maximum PSD in the second frequency band to detect the user's respiration signal from the second frequency band signal. As an example, the second frequency band signal may be divided into a 2-1 frequency band signal, a 2-2 frequency band signal, and a 2-3 frequency band signal according to a calculation of the PSD. In this instance, the signal detection unit 240 may compare a PSD of each of the 2-1 frequency band signal, the 2-2 frequency band signal, and the 2-3 frequency band signal, and detect the frequency band signal with the maximum PSD as the user's respiration signal.

As described above, the signal detection unit 240 may detect the user's heartbeat signal from the first frequency band signal according to a maximum PSD value of the first frequency band signal. Also, the signal detection unit 240 may detect the user's respiration signal from the second frequency band signal according to a maximum PSD value of the second frequency band signal. Specifically, the user's heartbeat signal or respiration signal may be detected with only an operation of calculating the PSD. Accordingly, the heartbeat signal or the respiration signal may be more accurately detected from the user's biosignal with only a simple configuration and operation.

The tracking unit 250 verifies whether the heartbeat signal or the respiration signal detected by the signal detection unit 240 corresponds to an accurate heartbeat signal or and accurate respiration signal of the user. Also, the tracking unit 250 may perform the verification by comparing the detected heartbeat signal or respiration signal with the user's previously detected heartbeat signal or respiration signal.

More specifically, the tracking unit 250 compares a PSD value of the heartbeat signal with a first threshold value. Here, the first threshold value may be set according to a PSD value of the user's previously detected heartbeat signal. As an example, the first threshold value may be set to a value corresponding to about 80% of the PSD value of the previously detected heartbeat signal.

Unless a human being is dead, a heart rate may never stop. Specifically, the heartbeat signal maintains a constant power at all times. Therefore, when the PSD of the detected heartbeat signal has a similar value with the PSD of the previously detected heartbeat signal within a predetermined error range, the tracking unit 250 may verify that the heartbeat signal corresponds to a normally detected heartbeat signal. Also, when the PSD of the detected heartbeat signal has a much smaller value than the PSD of the previously detected heartbeat signal, the tracking unit 250 may determine the detected heartbeat signal does not correspond to an accurate heartbeat signal of the user, but to a noise signal.

The tracking unit 250 compares the PSD value of the detected heartbeat signal with the first threshold value, and authenticates the detected heartbeat signal as the user's accurate heartbeat signal when the PSD value of the detected heartbeat signal is greater than or equal to the first threshold value. As described above, the first threshold value may be set to a value corresponding to about 80% of the PSD value of the previously detected heartbeat signal. Also, the first threshold value may be set to various values depending on a decision of those in the related arts.

When the PSD value of the detected heartbeat signal is less than the first threshold value, the tracking unit 250 may determine the detected heartbeat signal does not correspond to a heartbeat signal of the user, but a noise signal, and thereby generate an alarm signal and provide the user with the generated alarm signal, to induce the user to perform a re-measurement.

Also, when the PSD value of the detected heartbeat signal is less than the first threshold value, the tracking unit 250 may determine the user's heart activity is abnormal and thereby generate an alarm signal and provide the user with the generated alarm signal, to inform that the user may be in a dangerous health state. Also, the tracking unit 250 may transmit the alarm signal to a remote medical organization server or a family doctor's terminal using the communication unit 260.

When verifying the heartbeat signal, the tracking unit 250 compares a PSD value of the respiration signal with a second threshold value. Here, the second threshold value may be set according to a PSD value of the user's previously detected respiration signal. As an example, the second threshold value may be set to a value corresponding to 80% of the PSD value of the previously detected respiration signal. Also, the second threshold value may be set to various values depending on a decision of those in the related arts.

When the PSD value of the detected respiration signal is greater than or equal to the second threshold value, the tracking unit 250 authenticates the detected respiration signal as an accurate respiration signal of the user. However, when the PSD value of the detected respiration signal is less than the second threshold value, the tracking unit 250 may determine the user is in a breathless state.

Unlike a heart rate, a user may temporarily stop respiring. Therefore, in this instance, the tracking unit 250 may determine the user is in the breathless state and may induce the user to perform a re-measurement. However, when the breathless state is maintained, the tracking unit 250 may generate an alarm signal to inform that the user may be in a breathless state and transmit the generated alarm signal to a remote medical organization server or a family doctor's terminal using the communication unit 260.

As described above, the tracking unit 250 may include a memory 280 to maintain the PSD value of the user's previously detected heartbeat signal or respiration signal.

The communication unit 260 transmits the user's detected heartbeat signal or respiration signal to a predetermined server or a terminal. As an example, the communication unit 260 may transmit the heartbeat signal or the respiration signal to the predetermined server or the terminal that is located in a local area. Also, as described above, the communication unit 260 may transmit the heartbeat signal or the respiration signal to a remote medical organization server or a family doctor's terminal. To transmit the heartbeat signal or the respiration signal to an external server or an external terminal, the communication unit 260 may include all local communication modules that support various types of local area communications, such as Bluetooth, ZigBee, and the like. Also, when the biosignal detection apparatus 200 is included in a predetermined mobile communication terminal, the communication unit 260 may be constructed as a mobile communication module of the mobile communication terminal, such as a Code Division Multiplexing Access (CDMA) module, a High Speed Downlink Packet Access (HSDPA) module, a Wireless Broadband Internet (WiBro) module, and the like.

The display 270 may display the heartbeat signal or the respiration signal for the user. To display the heartbeat signal or the respiration signal, the display 270 may include a Super Twisted Nematic (STN) liquid crystal display (LCD) a thin film transistor (TFT) LCD, an organic electroluminescent LCD, and the like. Also, the display 270 may include the LCDs as described above, and also may include a Cathode Ray Tube (CRT), a Plasma Display Panel (PDP), and the like. However, it is not limited thereto.

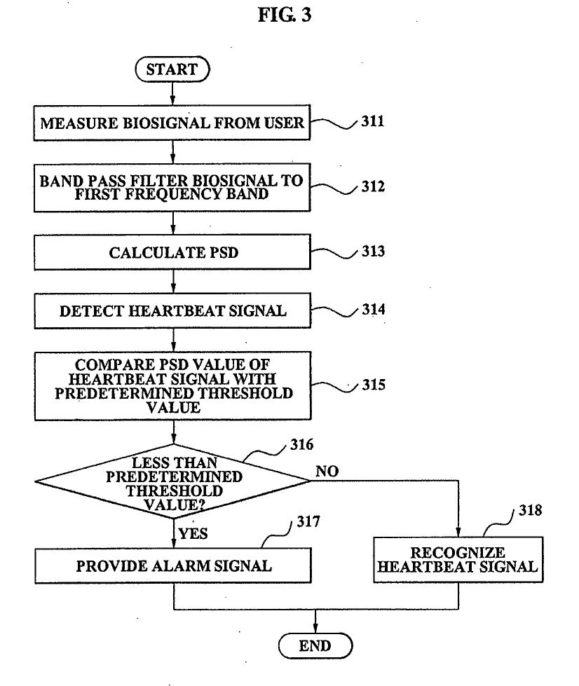

A configuration of a biosignal detection apparatus according to an exemplary embodiment and a method of detecting a biosignal according to the configuration have been described with FIGS. 1 and 2. Hereinafter, a method for detecting a heartbeat signal, a method of detecting a respiration signal, and a method for detecting a heartbeat signal and a respiration signal, which are performed by the biosignal detection apparatus, according to an exemplary embodiment will be described with reference to FIGS. 3 through 5.

FIG. 3 is a flowchart illustrating a method of detecting a biosignal according to an exemplary embodiment.

The biosignal detection method according to the present exemplary embodiment detects a user's heartbeat signal. In operation 311, a biosignal detection apparatus according to the present exemplary embodiment measures a biosignal from a user. Here, the biosignal detection apparatus may wirelessly measure the biosignal from the user in a local area via either a Doppler radar sensor, or a piezoelectric sensor and an RF sensor.

In operation 312, the biosignal detection apparatus band pass filters the biosignal to a first frequency band. Here, the first frequency band may be set to be within the range of about 1.0 Hz to about 1.5 Hz which corresponds to a human being's normal heartbeat frequency band. Also, the first frequency band may be set to a predetermined band based on a heart rate that is measured from the user for a predetermined period of time when the user is at rest.

In operation 313, the biosignal detection apparatus calculates a PSD of the band pass filtered signal to the first frequency band. In operation 314, the biosignal detection apparatus detects a frequency band signal with a maximum PSD in the first frequency band as the user's heartbeat signal.

In operation 315, the biosignal detection apparatus compares a PSD value of the heartbeat signal with a predetermined threshold value. Also, in operation 315, the predetermined threshold value may be set to a value corresponding to 80% of a previously measured PSD value of the user's heartbeat signal. When the PSD value of the heartbeat signal is less than the predetermined threshold value as a result of the comparison in operation S316, the biosignal detection apparatus provides an alarm signal to ask the user for a re-measurement in operation 317.

In operation 317, the biosignal detection apparatus may determine the user's heartbeat is abnormal and thereby generate the alarm signal to inform that the user may be in a dangerous health state and transmit the generated alarm signal to a remote medical organization server or a family doctor's terminal.

When the PSD value of the heartbeat signal is greater than or equal to the predetermined threshold value as a result of the comparison in operation 316, the biosignal detection apparatus determines the heartbeat signal, which is detected in operation 314, corresponds to an accurate heartbeat signal of the user in operation 318, and displays the heartbeat signal on a predetermined display for the user.

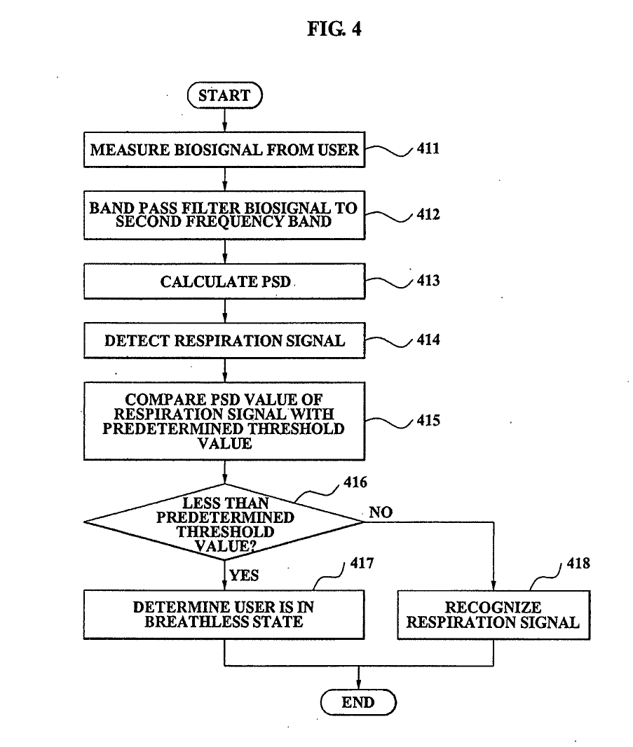

FIG. 4 is a flowchart illustrating a method of detecting a biosignal according to another exemplary embodiment.

The biosignal detection method according to the present exemplary embodiment detects a user's respiration signal. In operation 411, a biosignal detection apparatus according to the present exemplary embodiment measures a biosignal from a user. Here, the biosignal detection apparatus may wirelessly measure the biosignal from the user in a local area via either a Doppler radar sensor, or a piezoelectric sensor and an RF sensor. But it is not limited thereto.

In operation 412, the biosignal detection apparatus band pass filters the biosignal to a second frequency band. Here, the second frequency band may be set to be within the range of about 0.2 Hz to about 0.35 Hz which corresponds to a human being's normal respiration frequency band. Also, the second frequency band may be set to a predetermined band based on a respiration rate that is measured from the user for a predetermined period of time when the user is at rest.

In operation 413, the biosignal detection apparatus calculates a PSD of the band pass filtered signal to the second frequency band. In operation 414, the biosignal detection apparatus detects a frequency band signal with a maximum PSD in the second frequency band as the user's respiration signal.

In operation 415, the biosignal detection apparatus compares a PSD value of the respiration signal with a predetermined threshold value. Also, in operation 415, the threshold value may be set to a value corresponding to 80% of a previously measured PSD value of the user's respiration signal, for example. When the PSD value of the respiration signal is less than the predetermined threshold value as a result of the comparison in operation S416, the biosignal detection apparatus determines the user is in a breathless state in operation 417.

Also, in operation 417, the biosignal detection apparatus may provide an alarm signal to ask the user who is in the breathless state for a re-measurement. Also, when the user's breathless state is maintained, the biosignal detection apparatus may generate an alarm signal to inform that the user may be in a dangerous health state, and transmit the generated alarm signal to a remote medical organization server or a family doctor's terminal.

When the PSD value of the respiration signal is greater than or equal to the predetermined threshold value as a result of the comparison in operation 416, the biosignal detection apparatus determines the respiration signal, which is detected in operation 414, corresponds to an accurate respiration signal of the user in operation 418, and displays the respiration signal on a predetermined display for the user.

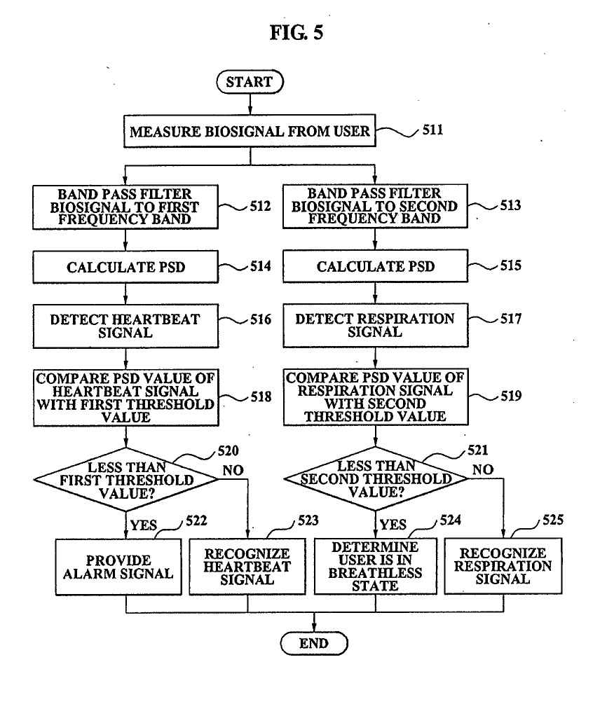

FIG. 5 is a flowchart illustrating a method of detecting a biosignal according to still another exemplary embodiment.

The biosignal detection method according to the present exemplary embodiment simultaneously detects a user's heartbeat signal and respiration signal. In operation 511, a biosignal detection apparatus according to the present exemplary embodiment measures a biosignal from a user. Here, the biosignal detection apparatus may wirelessly measure the biosignal from the user in a local area via either a Doppler radar sensor, or a piezoelectric sensor and an RF sensor.

In operation 512, the biosignal detection apparatus band pass filters the biosignal to a first frequency band. Here, the first frequency band may be set to be within the range of about 1.0 Hz to about 1.5 Hz which corresponds to a human being's normal heartbeat frequency band. Also, the first frequency band may be set to a predetermined band based on a heart rate that is measured from the user for a predetermined period of time when the user is at rest.

In operation 513, the biosignal detection apparatus band pass filters the biosignal to a second frequency band. Here, the second frequency band may be set to be within the range of about 0.2 Hz to about 0.35 Hz which corresponds to a human being's normal respiration frequency band. Also, the second frequency band may be set to a predetermined band based on a respiration rate that is measured from the user for a predetermined period of time when the user is at rest.

In operation 514, the biosignal detection apparatus calculates a PSD of the band pass filtered signal to the first frequency band. In operation S515, the biosignal detection apparatus calculates a PSD of the band pass filtered signal to the second frequency band.

In operation 516, the biosignal detection apparatus detects a frequency band signal with a maximum PSD in the first frequency band as the user's heartbeat signal. In operation 517, the biosignal detection apparatus detects a frequency band signal with a maximum PSD in the second frequency band as the user's respiration signal.

In operation 518, the biosignal detection apparatus compares a PSD value of the heartbeat signal with a first threshold value. Also, in operation 518, the first threshold value may be set to a value corresponding to 80% of a previously measured PSD value of the user's heartbeat signal, for example. When the PSD value of the heartbeat signal is less than the first threshold value as a result of the comparison in operation S520, the biosignal detection apparatus provides an alarm signal to ask the user for a re-measurement in operation 522. Also, in operation 522, the biosignal detection apparatus may determine the user's heartbeat is abnormal and thereby generate the alarm signal to inform that the user may be in a dangerous health state and transmit the generated alarm signal to a remote medical organization server or a family doctor's terminal.

When the PSD value of the heartbeat signal is greater than or equal to the first threshold value as a result of the comparison in operation 520, the biosignal detection apparatus determines the heartbeat signal, which is detected in operation 516, corresponds to an accurate heartbeat signal of the user in operation 523, and displays the heartbeat signal on a predetermined display for the user.

In operation 519, the biosignal detection apparatus compares a PSD value of the respiration signal with a second threshold value. Also, in operation 519, the second threshold value may be set to a value corresponding to about 80% of a previously measured PSD value of the user's respiration signal. When the PSD value of the respiration signal is less than the second threshold value as a result of the comparison in operation 521, the biosignal detection apparatus determines the user is in a breathless state in operation 524.

Also, in operation 524, the biosignal detection apparatus may provide an alarm signal to ask the user who is in the breathless state for a re-measurement. Also, when the user's breathless state is maintained, the biosignal detection apparatus may generate an alarm signal to inform that the user may be in a dangerous health state, and transmit the generated alarm signal to a remote medical organization server or a family doctor's terminal.

When the PSD value of the respiration signal is greater than or equal to the second threshold value as a result of the comparison in operation 521, the biosignal detection apparatus determines the respiration signal, which is detected in operation 517, corresponds to an accurate respiration signal of the user in operation 525, and displays the respiration signal on a predetermined display for the user.

The biosignal detection method according to each of the present exemplary embodiments has been described above with reference to FIGS. 3 through 5, and may include a configuration and operation of a biosignal detection apparatus according to the embodiment, which has been described above with reference to FIGS. 1 and 2.

The biosignal detection method according to the above-described exemplary embodiment may be recorded in computer-readable media including program instructions to implement various operations embodied by a computer. The media may also include, alone or in combination with the program instructions, data files, data structures, and the like. Examples of computer-readable media include magnetic media such as hard disks, floppy disks, and magnetic tape; optical media such as CD ROM disks and DVD; magneto-optical media such as optical disks; and hardware devices that are specially configured to store and perform program instructions, such as read-only memory (ROM), random access memory (RAM), flash memory, and the like. The media may also be a transmission medium such as optical or metallic lines, wave guides, and the like, including a carrier wave transmitting signals specifying the program instructions, data structures, and the like. Examples of program instructions include both machine code, such as produced by a compiler, and files containing higher level code that may be executed by the computer using an interpreter. The described hardware devices may be configured to act as one or more software modules in order to perform the operations of the above-described exemplary embodiments.

According to the above-described exemplary embodiments, there may be provided an apparatus and method of detecting a biosignal, which can measure a biosignal from a user using a radar, band pass filter the measured biosignal to a predetermined heartbeat frequency band, measure a PSD of the band pass filtered signal, and detect a frequency band signal with a maximum PSD as the user's heartbeat signal and thereby can reduce effects of motion artifacts of the user and also can more accurately and readily detect the user's heartbeat signal.

Also, according to the above-described exemplary embodiments, there may be provided an apparatus and method of detecting a biosignal, which can measure a biosignal from a user using a radar, band pass filter the measured biosignal to a predetermined respiration frequency band, measure a PSD of the band pass filtered signal, and detect a frequency band signal with a maximum PSD as the user's respiration signal, and thereby can reduce effects of motion artifacts of the user and also can more accurately and readily detect the user's respiration signal.

Also, according to the above-described exemplary embodiments, there may be provided an apparatus and method of detecting a biosignal, which can compare a PSD of each of a detected heartbeat signal and respiration signal with a previously measured PSD of each of the heartbeat signal and respiration signal, determine whether the detected heartbeat signal and the respiration signal correspond to accurate signals and thereby can secure a more accurate measurement with respect to the heartbeat signal and the respiration signal.

Although a few exemplary embodiments have been shown and described, the embodiment is not limited to the described exemplary embodiments. Instead, it would be appreciated by those skilled in the art that changes may be made to these exemplary embodiments without departing from the principles and spirit of the invention, the scope of which is defined by the claims and their equivalents.

1. A method of detecting a biosignal, the method comprising:
measuring a biosignal from a user;
band pass filtering the biosignal to a frequency band; and
calculating a power spectrum density (PSD) of the band pass filtered signal and detecting a frequency band signal with a maximum PSD.

2. The method of claim 1, wherein the biosignal is detected from the user using either a Doppler radar sensor, or a piezoelectric sensor and a radio frequency (RF) sensor.

3. The method of claim 1, wherein the frequency band is within a range of about 1.0 Hz to about 1.5 Hz.

4. The method of claim 1, wherein the frequency band is set to a predetermined band based on a heart rate that is measured from the user for a predetermined period of time when the user is at rest.

5. The method of claim 1, further comprising:
comparing a PSD value of the heartbeat signal with a predetermined threshold value; and
providing an alarm signal when the PSD value of the heartbeat signal is less than the predetermined threshold value as a result of the comparison.

6. The method of claim 5, wherein the predetermined threshold value is set to a value corresponding to about 80% of a previously measured PSD value of the user's heartbeat signal.

7. A method of detecting a biosignal, the method comprising:
measuring a biosignal from a user;
band pass filtering the biosignal to a frequency band; and
calculating a PSD of the band pass filtered signal and detecting a frequency band signal with a maximum PSD as a respiration signal of the user.

8. The method of claim 7, wherein the biosignal is detected from the user using either a Doppler radar sensor, or a piezoelectric sensor and an RF sensor.

9. The method of claim 7, wherein the frequency band is within the range of about 0.2 Hz to about 0.35 Hz.

10. The method of claim 7, wherein the frequency band is set to a predetermined band based on a respiration rate that is measured from the user for a predetermined period of time when the user is at rest.

11. The method of claim 7, further comprising:
comparing a PSD value of the respiration signal with a predetermined threshold value; and
determining the user is in a breathless state when the PSD value of the respiration signal is less than the predetermined threshold value as a result of the comparison.

12. The method of claim 11, wherein the predetermined threshold value is set to a value corresponding to about 80% of a previously measured PSD value of the user's respiration signal.

13. A method of detecting a biosignal, the method comprising:
measuring a biosignal from a user;
band pass filtering the biosignal to each of a first frequency band and a second frequency band, and detecting a first frequency band signal and a second frequency band signal;
calculating a PSD of the first frequency band signal and detecting a frequency band signal with a maximum PSD in the first frequency band as a heartbeat signal of the user; and
calculating a PSD of the second frequency band signal and detecting a frequency band signal with a maximum PSD in the second frequency band as a respiration signal of the user.

14. The method of claim 13, wherein the biosignal is detected from the user using either a Doppler radar sensor, or a piezoelectric sensor and an RF sensor.

15. The method of claim 13, wherein the first frequency band is within a range of about 1.0 Hz to about 1.5 Hz, and the second frequency band is within a range of about 0.2 Hz to about 0.35 Hz.

16. The method of claim 13, wherein each of the first frequency band and the second frequency band is set to a predetermined band based on a heart rate and a respiration rate that are measured from the user for a predetermined period of time when the user is at rest.

17. The method of claim 13, further comprising:
comparing a PSD value of the heartbeat signal with a first threshold value, and comparing a PSD value of the respiration signal with a second threshold value;
providing an alarm signal when the PSD value of the heartbeat signal is less than the first threshold value as a result of the comparison; and
determining the user is in a breathless state when the PSD value of the respiration signal is less than the second threshold value as a result of the comparison.

18. The method of claim 17, wherein the first threshold value is set to a value corresponding to 80% of a previously measured PSD value of the user's heartbeat signal, and the second threshold value is set to a value corresponding to 80% of a previously measured PSD value of the user's respiration signal.

19. A computer-readable recording medium storing a program for implementing the method according to claim 1.

20. An apparatus for detecting a biosignal, comprising:
a sensor unit measuring a biosignal from a user;
a filter unit band pass filtering the biosignal to each of a first frequency band and a second frequency band, and detecting a first frequency band signal and a second frequency band signal;
a PSD calculation unit calculating PSDs of the first frequency band signal and the second frequency band signal; and
a signal detection unit detecting a frequency band signal with a maximum PSD in the first frequency band as a heartbeat signal of the user, and detecting a frequency band signal with a maximum PSD in the second frequency band as a respiration signal of the user.

21. The apparatus of claim 20, wherein the sensor unit includes either a Doppler radar sensor, or a piezoelectric sensor and an RF sensor.

22. The apparatus of claim 20, wherein the first frequency band is within the range of about 1.0 Hz to about 1.5 Hz, and the second frequency band is within the range of about 0.2 Hz to about 0.35 Hz.

23. The apparatus of claim 20, wherein each of the first frequency band and the second frequency band is set to a predetermined band based on a heart rate and a respiration rate that are measured from the user for a predetermined period of time when the user is at rest.

24. The apparatus of claim 20, further comprising:
a tracking unit comparing a PSD value of the heartbeat signal with a first threshold value and generating an alarm signal when the PSD value of the heartbeat signal is less than the first threshold value as a result of the comparison, and comparing a PSD value of the respiration signal with a second threshold value and determining the user is in a breathless state when the PSD value of the respiration signal is less than the second threshold value as a result of the comparison.

25. The apparatus of claim 24, wherein the tracking unit sets the first threshold value to a value corresponding to about 80% of a previously measured PSD value of the user's heartbeat signal, and sets the second threshold value to a value corresponding to 80% of a previously measured PSD value of the user's respiration signal.
